(19) United States
(12) Patent Application Publication
(10) Pub. No.: US 2004/0016431 A1
(43) Pub. Date: Jan. 29, 2004

(54) Artificial ventilation apparatus

(76) Inventor: Laurent Preveyraud, Velizy (FR)

Correspondence Address: Air Liquide, Intellectual Property Department, Suite 1800, 2700 Post Oak Boulevard, Houston, TX, 77056, US

(21) Appl. No.: 10/332,740

(22) PCT Filed: Jun. 21, 2001
(86) PCT No.: PCT/FR01/01954

(30) Foreign Application Priority Data
Jul. 11, 2000 (FR) 00/09034

(51) Int. Cl. A61M 16/00

(52) U.S. Cl. 128/204.18; 128/204.21; 128/204.22

(57) ABSTRACT

The invention concerns a patient's ventilation with breathing mixture The installation comprises a ventilating assembly (10) for delivering said breathing mixture provided with a sensor for measuring the pressure (26) of the delivered mixture and a sensor for measuring the flow rate (28) of the delivered mixture; an inhalation conduit (12); and a device (30) for detecting accidental disconnection of the ventilation circuit comprising processing means (32, 34) for calculating, for each ventilating cycle, an apparent coefficient of the patient's compliance based on the measurements carried out by said pressure and flow rate sensors, using a predetermined formula; means (32, 34) for determining the sign of said compliance coefficient; means (32, 34) for comparing said compliance coefficient to a predetermined positive value; and means (40) for triggering a warning (42, 44) if the compliance coefficient is negative or if the compliance coefficient is higher than said predetermined value.

The subject of the present invention is an installation for ventilating a patient with breathing mixture.

More specifically, the invention relates to a system for detecting the accidental or inadvertent disconnection of the circuit connecting the ventilating machine to the patient.

The term ventilating installation refers not only to the actual installation for ventilating the patient, but also to the management or monitoring system which is often combined with the actual ventilating installation. This is because this system has its own means for detecting disconnection.

When using an installation for ventilating a patient in a hospital environment or at home, it is of course very important to provide a system for triggering an alarm should one or more of the elements forming the patient circuit connecting the ventilating machine to the patient's airways become disconnected.

Furthermore, it is known that such installations may operate in various modes. In the case of pressure ventilating, the pressure of the breathing mixture at the outlet of the machine can be held constant. In the volume operating mode, it is the flow rate of breathing mixture at the outlet of the ventilating machine which is controlled. It may for example be kept constant or decelerate, or change in some other way, the ventilator guaranteeing in all cases a certain volume of breathing mixture.

Depending on the case, and whether the ventilating is in pressure or volume mode, provision can be made that during the exhalation phase of a ventilating cycle, the pressure of the breathing mixture applied to the patient is equal to atmospheric pressure. Provision can also be made that, during this exhalation phase, the pressure of the breathing mixture is kept greater than atmospheric pressure, in order to maintain a certain pressure during this phase inside the pulmonary system of the patient. This second operating mode is called ventilating with positive exhalation pressure and denoted by the abbreviation Pₑₚ.

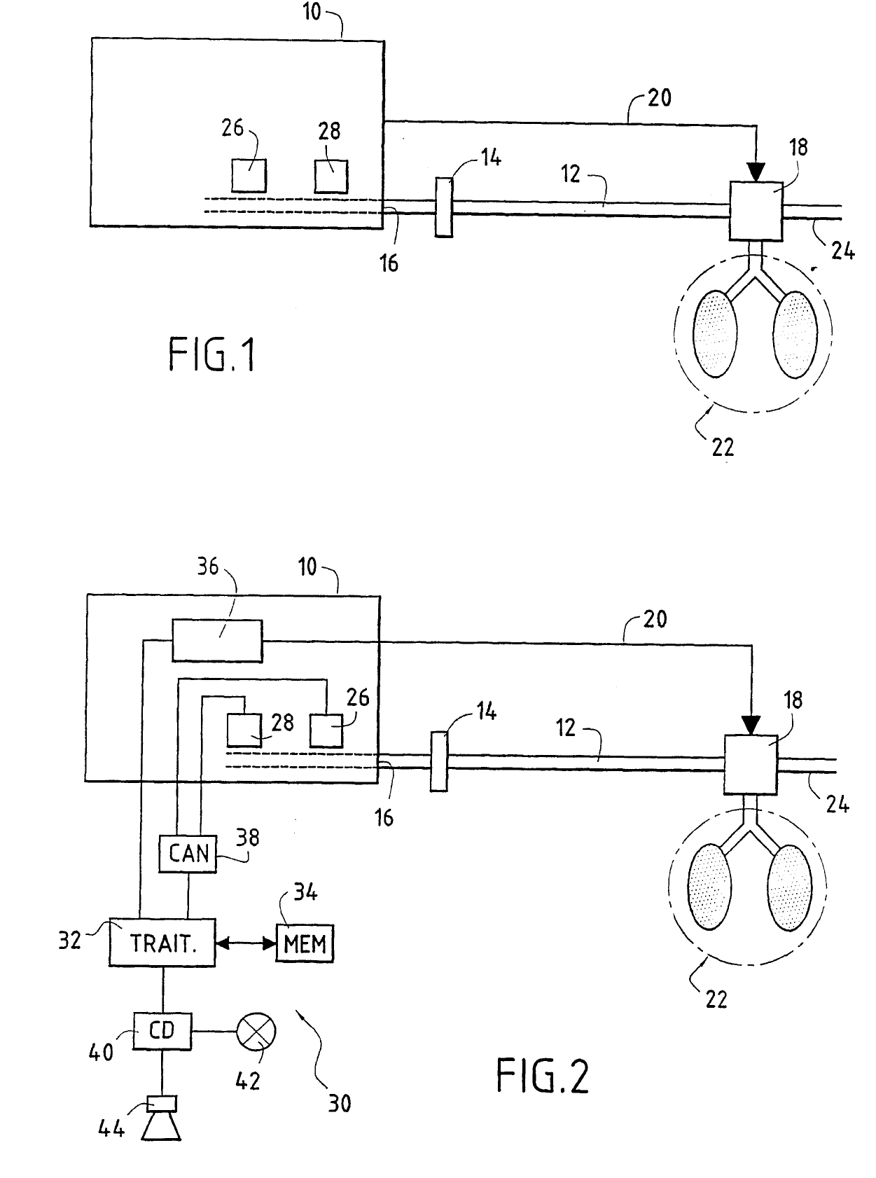

FIG. 1 shows, in a simplified manner, an installation of known type. The ventilating machine 10 is shown, to the outlet of which is connected the tube 12 of the patient circuit, this tube 12 preferably having a bacteriological filter 14 placed close to the outlet 16 of the ventilating machine 10. The tube 12 is connected to an exhalation valve 18, whose state is controlled at 20 by the machine 10. The exhalation valve 18 is connected to a mask which places the tube 12 in communication with the patient's pulmonary system 22. The exhalation valve 18 is, of course, also connected to a tube 24 for exhaled breathing mixture. In a machine 10 of this sort, a pressure measuring sensor 26 and a flow rate measuring sensor 28 mounted close to the outlet 16 are also found. These sensors are especially used in order to define the pressure or volume operating system.

In known systems, disconnection of the tube 12 going to the patient is usually detected by monitoring the crossing of a pressure threshold which may be adjustable and which takes into account the normal change in pressure during the phase of insufflating the patient with the breathing mixture. It is then the responsibility of the user to adjust this threshold properly so as to detect the disconnection of the tube 12 having the effect of interrupting the patient's ventilating. Adjustment of this threshold is made particularly tricky if the ventilator maintains a residual flow rate in the insufflation circuit during the exhalation phase. In this case, the presence of a residual pressure resulting from the pressure drop in the circuit on the patient's side and from the residual flow rate makes it less easy to adjust this threshold. This pressure drop in the circuit also has a drawback when the circuit 12 is disconnected during the phase of insufflating the patient with breathing mixture while the ventilating is taking place in pressure mode, since the pressure resulting therefrom may then go beyond the adjusted set threshold for minimum pressure, masking the disconnection of the patient from the machine.

It is understood that this mode of triggering an alarm in the case of accidental or inadvertent disconnection of the circuit delivering breathing mixture to the patient, on the one hand, involves a tricky adjustment and, on the other hand, may lead to inadvertent triggerings.

One object of the present invention is to provide an installation for ventilating a patient with breathing mixture, which has a device for triggering an alarm in the event of disconnection of the breathing-mixture delivery circuit which alleviates the drawbacks mentioned above.

To achieve this aim according to the invention, the installation for ventilating a patient with breathing mixture comprises a ventilating assembly for delivering said breathing mixture provided with a sensor for measuring the pressure of the delivered mixture and/or a sensor for measuring the flow rate of the delivered mixture;

an inhalation conduit; and

a device for detecting the accidental disconnection of the ventilating circuit comprising:

processing means for calculating for each ventilating cycle, an apparent compliance coefficient of the patient based on measurements carried out by said pressure and flow rate sensors, using a predetermined formula,

means for determining the sign of said compliance coefficient,

means for comparing said compliance coefficient with a predetermined positive value and

means for triggering an alarm if the compliance coefficient is negative or if the compliance coefficient is greater than said predetermined value.

It is understood that the device for triggering an alarm in the event of disconnection is based on measuring the apparent compliance coefficient of the patient. This apparent compliance coefficient either becomes negative, or it takes a very high value in the event of disconnection whether operating in pressure mode or volume mode or else with positive exhalation pressure. Thus the device for triggering an alarm is not dependent on the accurate adjustment of a reference pressure which is compared to a pressure within the breathing-mixture delivery duct.

In the case of operating in a mode without positive exhalation pressure, the predetermined formula is of the type:

$$C = \frac{V}{{P}_{finI} - {KQ}_{finI}}$$

in which V is the volume of breathing mixture delivered during an inhalation cycle, P_{finI} is the pressure of the breathing mixture delivered to the patient at the end of an inhalation cycle, Q_{finI} is the flow rate delivered at the end of an inhalation cycle and K is a constant.

In the case of operation with positive exhalation pressure, the predetermined formula is of the type:

$$C = \frac{V}{{P}_{finI} - {P}_{finE} - {KQ}_{finI}}$$

in which V is the volume of breathing mixture delivered during an inhalation cycle, P_{finI} is the pressure of the breathing mixture delivered to the patient at the end of an inhalation cycle, Q_{finI} is the flow rate at the end of an inhalation cycle, P_{finE} is the pressure at the end of an exhalation cycle and K is a constant.

Preferably, the predetermined apparent compliance coefficient is equal to 300 ml/cm H₂O.

Another object of the invention is to provide a method of detecting the accidental disconnection of the inhalation circuit of an installation for ventilating a patient with breathing mixture, comprising a ventilating assembly, the method comprising the following steps:

the pressure and/or the flow rate of breathing mixture is measured or determined at the outlet of the ventilating assembly for one ventilating cycle;

an apparent compliance coefficient of the patient is calculated, from said measurements, using a predetermined equation;

an alarm is triggered if the value of said compliance coefficient is negative or if it is greater than a predetermined value.

Other features and advantages of the invention will become better apparent on reading the following description of a preferred embodiment of the invention given by way of non-limiting example. The description refers to the appended figures, in which:

FIG. 1, already described, shows a standard ventilating installation; and

FIG. 2 shows a ventilating installation equipped with the device for detecting accidental or inadvertent disconnection.

In the present description, the term compliance of the patient or compliance coefficient refers to the elastic ability of the patient's lung which is subject to the ventilating, that is to say that it involves the coefficient which associates the volume of the lungs with the pressure of the breathing mixture introduced into the lungs.

With reference to FIG. 2, the ventilating installation and, more specifically, the device for triggering an alarm in the case of inadvertent disconnection of the ventilating duct, will be described. The triggering device 30 mainly consists of a processing unit 32 associated with a memory 34 for storing programs. The processing circuit 32 is connected to the main circuit 36 controlling the ventilating machine 10. The processing unit 32 is also connected to the pressure and flow rate sensors 26 and 28 via an analogue/digital converter 38. The processing circuits 32 are also connected to a control unit 40 intended to control the actuation of alarm means such as, for example, the light alarm 42 or the sound alarm 44.

Thus, as has already been explained, detection of the disconnection is based on calculating an apparent compliance coefficient generated from measurements carried out by the pressure and flow rate sensors based on pre-established formulae depending on the operating mode of the machine, that is to say depending on whether the machine operates with or without positive exhalation pressure. The memory 34 has subprograms for calculating the compliance coefficient and comparison subprograms. The function of the processing circuit 32 is mainly to calculate the compliance coefficient and to control the control circuit 40 in order to trigger the alarm if the situation so requires.

More specifically, the value of the calculated apparent compliance coefficient is compared to zero and to a positive predetermined value C_{M} which is preferably equal to 300 ml/cm of water, this value C being stored in the memory 34. If the calculated compliance coefficient is negative or if it is greater than the predetermined value C, the control circuit 40 is activated and the alarm is triggered. If the value of the calculated compliance coefficient is between these two values, the ventilating machine operates normally.

If the pressure P(t) at the outlet 16 of the ventilating machine is considered, it can be written as:

$$P ( t ) - P ( 0 ) = ( {R}_{1} + {R}_{2} + {R}_{3} + {R}_{4} ) \times Q ( t ) + \frac{V ( t ) - V ( 0 )}{C}$$

In this formula, P(0) and V(0) represent the initial pressure and volume of the breathing mixture.

P(t), Q(t) and V(t) represent the pressure and flow rate at the outlet 16 of the machine and the volume of breathing mixture delivered at time t, respectively.

R₁, R₂, R₃ and R₄ represent the hydraulic resistance of the patient, of the filter, of the duct for insufflating breathing mixture to the patient and of the exhalation valve, respectively. Finally, C represents the compliance of the patient. In practice, R₄ can be ignored.

At the end of the inhalation phase, and if the ventilating is carried out without Pₑₚ, the compliance coefficient C can be written:

$$C = \frac{V}{{P}_{FinI} - ( {R}_{1} + {R}_{2} + {R}_{3} ) \times {Q}_{FinI}} \qquad ( 1 )$$

If the installation operation is with Pₑₚ, we then have:

$$C = \frac{V}{{P}_{finI} - {P}_{FinE} - ( {R}_{1} + {R}_{2} + {R}_{3} ) \times {Q}_{finI}} \qquad ( 2 )$$

In these formulae, V represents the total volume of breathing mixture delivered, P_{FinI} and Q_{FinI} represent the pressure and the flow rate at the end of the inhalation phase and P_{finE} represents the pressure at the end of the exhalation phase.

It is understood that by measuring P and Q at suitable times, it is possible to calculate the compliance coefficient of the patient.

Most specifically, it is possible to calculate the apparent compliance coefficient of the patient which will be the effective compliance of the patient if there is no disconnection of the ventilating machine and if the hydraulic resistance of the patient is known, and which will otherwise be an “apparent” compliance coefficient.

The processing circuit calculates the compliance coefficient from formula (1) or (2) and from pressure and flow rate measurements using subprograms stored in the memory, these programs making it possible to use the two formulae depending on the operating mode of the machine. It should be noted that if the ventilating is carried out without Pₑₚ, but the value of P_{finE} is not zero, the formula (2) can be used.

As has already been indicated, the alarm is triggered if the compliance coefficient is negative or if it becomes greater a predetermined value C_{M}.

It can be confirmed that, whether operating in pressure or volume mode, with Pₑₚ or without Pₑₚ, in the event of disconnection either from the outlet of the machine, or from one of the orifices of the exhalation valve, the apparent compliance coefficient becomes either very large, or negative.

In the case of pressure ventilating, without or without Pₑₚ, in the event of disconnection from the exhalation valve, the volume V delivered during the inhalation phase is large because of the pressure adjustment and the pressure at the end of phase P_{Fin}I is almost equal to the pressure drop (R₂+R₃)Q_{FinI}. The denominator of the fraction giving the compliance is virtually zero while the numerator is large. The calculation therefore provides a “non-physiological” apparent compliance value.

If disconnection occurs at the outlet of the exhalation machine, the hydraulic resistances R₁, R₂ and R₃ are removed. The pressure P_{Fin}I therefore becomes very small. In the formula (1) or (2), the denominator becomes negative and of course it is necessarily the same for the apparent compliance coefficient for the patient.

In the case of volumetric ventilating, if the circuit is disconnected at the exhalation value, the flow rate remains constant and the pressure at the end of the inhalation phase is equal to the product of the flow rate times the pressure drop of the filter and of the duct. Since there is no accumulation of a volume of breathing mixture by the patient, there is no pressure rise. The denominator of the fraction of formulae (1) and (2) becomes close to zero. The denominator may even become negative through participation of the pressure drops of the ventilating circuit. Since the volume V remains normal (numerator), the apparent compliance coefficient therefore becomes either very large, or negative. If the outlet of the machine becomes disconnected, the same situation occurs as for the pressure ventilating and the coefficient C becomes negative.

1. An installation for ventilating a patient with breathing mixture comprising:
a ventilating assembly for delivering said breathing mixture provided with a sensor for measuring the pressure of the delivered mixture and/or a sensor for measuring the flow rate of the delivered mixture;
an inhalation conduit; and
a device for detecting the accidental dis-connection of the ventilating circuit comprising:
processing means for calculating, for each ventilating cycle, an apparent compliance coefficient of the patient based on measurements carried out by said pressure and flow rate sensors, using a predetermined formula,
means for determining the sign of said compliance coefficient,
means for comparing said compliance coefficient with a predetermined positive value and
means for triggering an alarm if the compliance coefficient is negative or if the compliance coefficient is greater than said predetermined value.

2. The ventilating installation operating in a mode without positive exhalation pressure as claimed in claim 1, characterized in that said predetermined formula is of the type:
$$C = \frac{V}{{P}_{finI} - {KQ}_{finI}}$$
in which V is the volume of breathing mixture delivered during an inhalation cycle, P_{finI} is the pressure delivered at the end of an inhalation cycle, Q_{finI} is the flow rate delivered at the end of an inhalation cycle and K is a constant.

3. The ventilating installation operating in a mode with positive exhalation pressure as claimed in claim 1, characterized in that said predetermined formula is of the type:
$$C = \frac{V}{{P}_{finI} - {P}_{finE} - {KQ}_{finI}}$$
in which V is the volume of breathing mixture delivered during an inhalation cycle, P_{finI} is the pressure at the end of an inhalation cycle, Q_{finI} is the flow rate at the end of an inhalation cycle, P_{finE} is the pressure at the end of an exhalation cycle and K is a constant.

4. The ventilating installation as claimed in any one of claims 1 to 3, characterized in that said predetermined value is about 300 expressed in ml/cm of H₂O.

5. A method of detecting the accidental disconnection of the inhalation circuit of an installation for ventilating a patient with breathing mixture, comprising a ventilating assembly, comprising the following steps:
the pressure and/or the flow rate of breathing mixture is measured or determined at the outlet of the ventilating assembly for one ventilating cycle;
an apparent compliance coefficient of the patient is calculated, from said measurements, using a predetermined equation;
an alarm is triggered if the value of said compliance coefficient is negative or if it is greater than a predetermined value.

6. The method as claimed in claim 5, characterized in that said predetermined formula is of the type:
$$C = \frac{V}{{P}_{finI} - {KQ}_{finI}}$$
in which V is the volume of breathing mixture delivered during an inhalation cycle, P_{finI} is the pressure delivered at the end of an inhalation cycle, Q_{finI} is the flow rate delivered at the end of an inhalation cycle and K is a constant.

7. The method as claimed in claim 5, characterized in that said predetermined formula is of the type:
$$C = \frac{V}{{P}_{finI} - {P}_{finE} - {KQ}_{finI}}$$
in which V is the volume of breathing mixture delivered during an inhalation cycle, P_{finI} is the pressure at the end of an inhalation cycle, Q_{finI} is the flow rate at the end of an inhalation cycle, P_{finE} is the pressure at the end of an exhalation cycle and K is a constant.

8. The method as claimed in any one of claims 5 to 7, characterized in that said predetermined value is about 300 expressed in ml/cm of H₂O.
